(19) United States
(12) Patent Application Publication
(10) Pub. No.: US 2016/0143972 A1
(43) Pub. Date: May 26, 2016

(54) METHOD AND APPARATUS FOR PREPARING A SOLID FORM OF CANNABINOID

(71) Applicants: Russell Hobart Stebbins, Clyde Hill, WA (US); Derick Scott Anderson, Seattle, WA (US)

(72) Inventors: Russell Hobart Stebbins, Clyde Hill, WA (US); Derick Scott Anderson, Seattle, WA (US)

(73) Assignee: Cannamark Inc., Clyde Hill, WA (US)

(21) Appl. No.: 14/949,546

(22) Filed: Nov. 23, 2015

Related U.S. Application Data
(60) Provisional application No. 62/082,690, filed on Nov. 21, 2014.

(51) Int. Cl. A61K 36/185; A61K 9/16

(52) U.S. Cl. CPC A61K 36/185; A61K 9/1682; A61K 9/1652

(57) ABSTRACT

Disclosed is a method of preparing a solid form of a cannabinoid. The solid form of the Cannabinoid may be substantially soluble in an aqueous solution. The method may include dissolving each of a Cannabinoid and one or more emulsifying agents into one or more solvents to obtain one or more combined solutions. Further, the method may include separating the one or more solvents from the one or more combined solutions to obtain the solid form of the cannabinoid.

### RELATED APPLICATIONS

The present application claims priority to U.S. Provisional Patent Application No. 62/082,690, filed Nov. 21, 2014, entitled “Dry Solid Distillate Synthesis”, the disclosure of which is incorporated herein by reference in its entirety.

### FIELD

The present invention generally relates to drugs. More specifically, the present invention relates to methods and apparatus for preparing solid form of cannabinoids.

### BACKGROUND

Cannabinoids are compounds derived from Cannabis, a flowering plant that includes three species, namely, sativa, indica, and ruderalis. Cannabis has widely been used as a recreational drug and as a medication to alleviate several conditions. Some examples of medicinal use of cannabis include treatment of glaucoma, cramps, migraines, convulsions, appetite stimulation, spasticity associated with multiple sclerosis, fibromyalgia and nausea. Additionally, Cannabis has also been commonly used as an ingredient in popular foods, such as, pot brownies, cannabis beverages and cannabis candies.

Cannabis may be consumed in a variety of ways. For example, Cannabis may be ingested by smoking, inhaling vapors, eating foods with Cannabis cooked or baked into them, placing drops of tinctures or sublingual preparations directly under the tongue, on the skin, or in foods or drinks, applying topical or transdermal applications to the skin, drinking cannabis infused beverages, and smoking cannabis concentrates or eating the resin made from cannabis plant. However, these methods of consumption do not offer a user an accurate measure of Cannabinoids that they are consuming. Further, there are limited methods, if any, that allow the user to consume powdered preparations of cannabis such as drink mixes. Moreover, Cannabinoids are generally hydrophobic in nature. As a result, they exhibit poor dissolution in aqueous environments, such as the gastrointestinal tract, leading to insufficient systemic absorption. Consequently, bioavailability of Cannabinoids may be very low.

Accordingly, there is a need for improved formulations of Cannabinoids that may exist in a solid form and provide substantial dissolution in aqueous solutions.

### SUMMARY

Disclosed is a method of preparing a solid form of a cannabinoid. The solid form of the cannabinoid may be substantially soluble in an aqueous solution. The method may include dissolving each of a cannabinoid and one or more emulsifying agents into one or more solvents to obtain one or more combined solutions. Further, the method may include separating the one or more solvents from the one or more combined solutions to obtain the solid form of the cannabinoid.

Also disclosed is a method of preparing a powder form of a cannabinoid. The method may include dissolving the cannabinoid into a carrier solvent of one or more solvents to obtain a cannabinoid solution. Further, the cannabinoid solution may be mixed to obtain a homogenized cannabinoid solution. Subsequently, the one or more emulsifying agents may be dissolved into an aqueous solvent of the one or more solvents to obtain an emulsifier solution. Thereafter, the emulsifier solution may be mixed to obtain a homogenized emulsifier solution. Subsequently, each of the homogenized cannabinoid solution and the homogenized emulsifier solution may be mixed to obtain a homogenized combined solution. Further, each of the carrier solvent and the aqueous solvent may be evaporated from the homogenized combined solution to obtain a solid form of the cannabinoid. Subsequently, the solid form of the cannabinoid may be milled to obtain the powder form of cannabinoid.

### BRIEF DESCRIPTION OF DRAWINGS

FIG. 1 illustrates a flow chart of a method for preparing a solid form of a Cannabinoid according to some embodiments.

FIG. 2 illustrates a flow chart of a method for preparing a powder form of a Cannabinoid according to some embodiments.

FIG. 3 illustrates a flow chart of producing a solid form of an essential oil according to some embodiments.

FIG. 4 illustrates an apparatus for preparing a powder form of a cannabinoid according to some embodiments.

### DETAILED DESCRIPTION

Overview:

According to the present disclosure, an aqueous cannabinoid emulsification may be prepared using emulsifying agents such as glycoprotein and polysaccharide complexes. As a result, it may be possible to bind extracted cannabinoid concentrates with these complexes in order to create a powdered cannabinoid mixture which has numerous applications. For example, powdered Cannabinoids may be used in the creation of powdered drink additives and cooking/baking additives. Additionally, the Cannabinoids may be homogenously distributed throughout an aqueous solution.

In some embodiments, the emulsifying agent may be completely homogenized with water at a temperature between 0 and 100 degrees Celsius to obtain an emulsifying solution. Subsequently, a Cannabinoid solution may be obtained by dissolving Cannabinoid extracts in a solvent such as ethanol. Further, the Cannabinoid solution may be completely homogenized. Thereafter, the emulsifying solution and the Cannabinoid solution may be combined using a homogenization machine at a temperature between 0 and 100 degrees Celsius. Once the two solutions are homogenized, the liquid may be evaporated using, for example, air evaporation or a spray dryer. Consequently, condensed solid may be obtained. Subsequently, the condensed solid may be processed through a mill in order to create a uniform powder containing Cannabinoids.

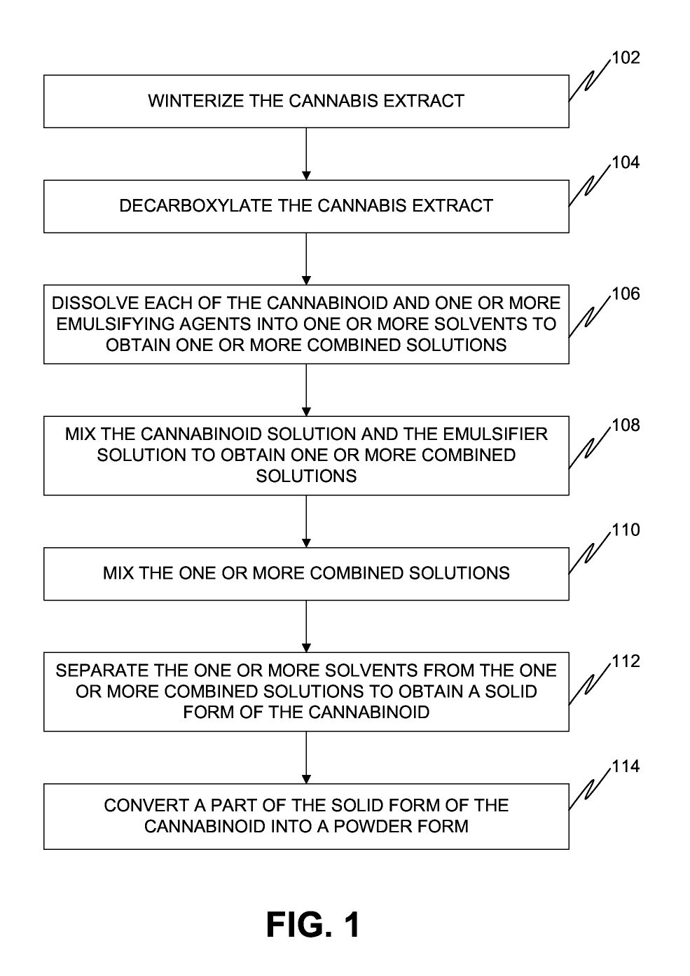

According to the present disclosure, a method of preparing a solid form of a Cannabinoid is disclosed as illustrated in FIG. 1. The Cannabinoid may include one or more of Cannabidiol (CBD), Cannabinol (CBN) and Δ9-tetrahydrocannabinol (THC).

Further, in some embodiments, the solid form of the Cannabinoid may be substantially soluble in an aqueous solution.

Further, in some embodiments, the Cannabinoid may be included in a cannabis extract obtained from a cannabis plant. Accordingly, the method may include a step 102 for winterizing the cannabis extract. Additionally, the method may include a step 104 decarboxylating the cannabis extract. Accordingly, the cannabis extract may retain a natural terpene profile or have a terpene profile added to it.

Further, the method may include a step 106 of dissolving each of the Cannabinoid and one or more emulsifying agents into one or more solvents to obtain one or more combined solutions. In some embodiments, the one or emulsifying agents may include one or more of natural gum, β-Glucan and cyclic-β-Glucan. Further, in some embodiments, the one or more emulsifying agents may include gum arabic.

In some embodiments, a weight ratio of the cannabis extract to the one or more emulsifying agents may be a minimum of 4:1. In other embodiments, the weight ratio may be 1:1. Furthermore, in some embodiments, the weight ratio may be 1:4. Generally, the cannabis extract may comprise 25% to 90% of the cannabinoid by weight. Accordingly, in some embodiments, a weight ratio of the Cannabinoid to the one or more emulsifying agents may be in a range of 1 to 16.

In some embodiments, the step 106 of dissolving may include a step 106a (not shown in figure) of dissolving the Cannabinoid into a carrier solvent of the one or more solvents to obtain a Cannabinoid solution. In some embodiments, the carrier solvent may be characterized by each of miscibility with water and ability to dissolve the cannabinoid. Further, in some embodiments, the carrier solvent may include an alcohol. Furthermore, in some embodiments, the carrier solvent may be a short chained alcohol consisting of 1-4 carbon atoms. Additionally, in some embodiments, a temperature of the carrier solvent may be in a range of 0 to 100 degrees Celsius. In some embodiments, a temperature of the carrier solvent may be in a range of 0 to 120 degrees Celsius.

Further, the step 106 of dissolving may include a step 106b (not shown in figure) of dissolving the one or more emulsifying agents into an aqueous solvent of the one or more solvents to obtain an emulsifier solution. In some embodiments, a temperature of the aqueous solvent may be in a range of 0 to 100 degrees Celsius.

Additionally, in some embodiments, the method may include a step 108 of mixing each of the Cannabinoid solution and the emulsifier solution to obtain the one or more combined solutions. In some embodiments, each of Cannabinoid solution and the emulsifier solution may be slowly added together into a container. Additionally, in some embodiments, the method may further include a step 110 of mixing the one or more combined solutions.

Further, the method may include a step 112 of separating the one or more solvents from the one or more combined solutions to obtain the solid form of the cannabinoid. In some embodiments, the separating may include one or more of evaporating and drying. Accordingly, in some embodiments, the step 112 may include a step 112a (not shown in figure) of evaporating the carrier solvent from the one or more combined solutions. Accordingly, in some embodiments, the carrier solvent separated through evaporation may be recovered and recycled for subsequent use. Further, the step 112 may include a step 112b (not shown in figure) of evaporating the aqueous solvent from the one or more combined solutions.

In some embodiments, the separating may be performed using mechanical evaporation equipment such as, for example, a spray dryer. Further, a nozzle of the spray dryer may be configured to create an average particle size of less than 8,000 microns. In some embodiments, the nozzle of the spray dryer may be configured to create an average particle size of less than 5,000 microns. Further, in some embodiments, the nozzle of the spray dryer may be configured to create an average particle size of less than 500 microns.

In some embodiments, the solid form of the Cannabinoid may include a powder form of the cannabinoid. Accordingly, in some embodiments, the method may further include a step 114 of converting a part of the solid form of the Cannabinoid into a powder form of the cannabinoid. Furthermore, in some embodiments, a weight ratio of the Cannabinoid and the one or more emulsifying agents in the solid form of Cannabinoid may be in a range of 0.05 to 25.

Further, in case the evaporating is performed using means other than spray drying, in some embodiments, the solid form of the Cannabinoid may need to be milled to an average particle size of less than 8000 microns. Accordingly, in some embodiments, the converting may include milling. Additionally, an average particle size of the powder form of the Cannabinoid may be in a range of 500 microns to 8000 microns. In some embodiments, the average particle size of the powder form may be below 1 micron. In some other embodiments, the average particle size of the powder form may be the smallest size obtainable by available technology, such as, for example, 141 nm.

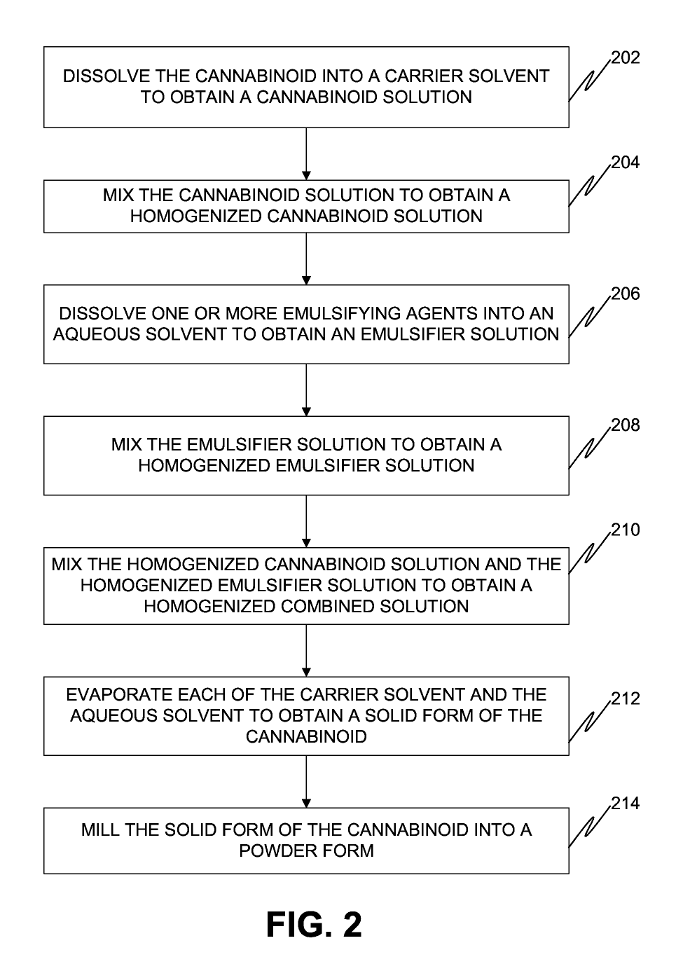

According to another embodiment, a method of preparing a powder form of a cannabinoid is disclosed as illustrated by FIG. 2. The method may include a step 202 of dissolving the Cannabinoid into a carrier solvent of one or more solvents to obtain a Cannabinoid solution. Further, the Cannabinoid solution may be mixed to obtain a homogenized Cannabinoid solution at step 204. Subsequently, at step 206, the one or more emulsifying agents may be dissolved into an aqueous solvent of the one or more solvents to obtain an emulsifier solution. Thereafter, at step 208, the emulsifier solution may be mixed to obtain a homogenized emulsifier solution. Subsequently, at step 210, each of the homogenized Cannabinoid solution and the homogenized emulsifier solution may be mixed to obtain a homogenized combined solution. Further, at step 212, each of the carrier solvent and the aqueous solvent may be evaporated from the homogenized combined solution to obtain a solid form of the cannabinoid.

Subsequently, at step 214, the solid form of the Cannabinoid may be milled to obtain the powder form of cannabinoid.

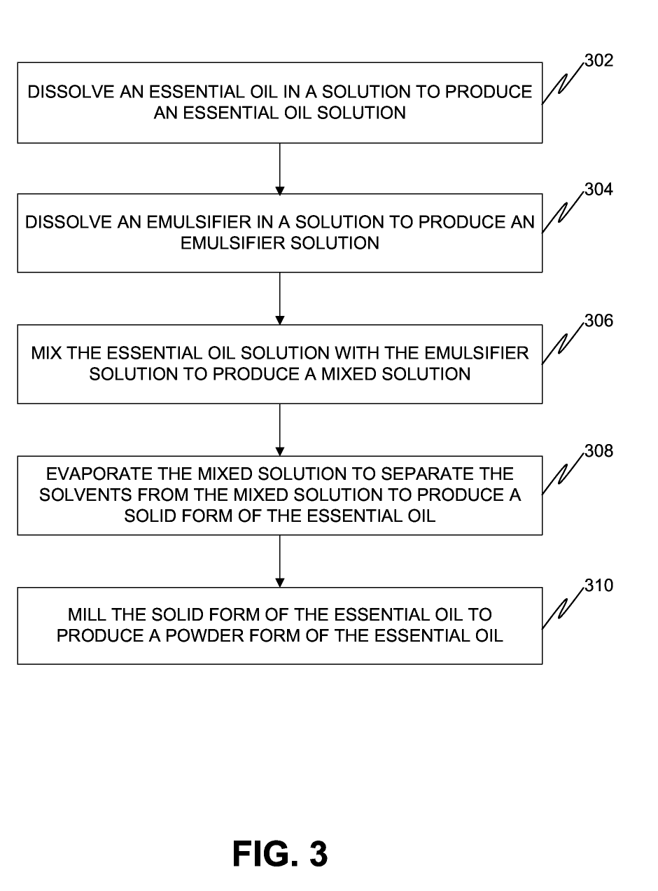

FIG. 3 illustrates a flow chart of a method of producing a solid form of an essential oil.

The solid form of the essential oil may, in some embodiments, be soluble in water. In general, the essential oils may be characterized by non-miscibility with an aqueous solution. Examples of essential oils include, but are not limited to, terpenes, terpenoids, palm oils, omega-3 oils, lavender, peppermint, tea tree oil, eucalyptus, colorings and flavorings. In some embodiments, the essential oil may include a Cannabinoid such as, for example, Cannabidiol (CBD), Cannabinol (CBN) or Δ9-tetrahydrocannabinol (THC). In some embodiments, the method may be applicable in the production of resins and tinctures.

At step 302, the essential oil may be dissolved in a solution to produce an essential oil solution. In some embodiments, the solution may include a substrate. Further, in some embodiments, the substrate may be a food product such as, for example, sugar and dairy products. Accordingly, the substrate may include, but is not limited to, one or more of alcohol and water soluble forms of at least one of the following: a) carbohydrates and simple sugars such as, for example, glucose, sucrose, fructose, dextrose, cane sugar, molasses, galactose, maltodextrin, monosaccharides, grape sugar, plant juices, fruit sugars occurring naturally, root vegetable sugars and starches, agave, and honey; b) artificial sweeteners such as, for example, erythritol, appertain, stevia, xylitol, aspartame, sucralose, neotame, acesulfame potassium (Ace-K), saccharin and advantame; c) confectioner's sugar, granular sugars, sugar products, juices, fruits, concentrates, vegetable solids and soy; d) natural sugar substitutes such as, for example, sorbitol and xylitol, which are found in berries, fruit, vegetables and mushrooms; e) dairy such as, for example, milk, cream and cheeses. In general, the substrate may include any substance capable of acting as a colloid within the solution or capable of being suspended in an agitated solution.

In some embodiments, agitation of a solution may include application of heat without exceeding the specific denaturing temperatures or degradation temperatures of the constituents of the solution. A solution may be agitated via use of a hot plate with a magnetic stirring bar, through the use of recirculating pumps, through the use of aeration techniques, through the use of variable heat apparatus causing flow via temperature gradient, manual mechanical mixing through the use of a spoon, ladle, spatula or any other physical mixing device. Further, tools such as evaporator pans, blenders, any form of, stock pot, beaker or other fluid containing vessel may be used for containing and providing a location for agitation. Agitation of the solution through the use the aforementioned is necessary bringing the substrate into homogeneous solution with the fluid.

In some embodiments, each of the essential oil and the substrate may be dissolved into the solution together. In some other embodiments, the substrate may be separately dissolved in the solution followed by agitation of the solution. Subsequently, the essential oil may be dissolved into the solution including the substrate.

In some embodiments, an emulsifier may be necessary under conditions in which the solid form of the essential oil is intended to be dissolvable in an aqueous solution at room temperature. Accordingly, at step 304, an emulsifier may be dissolved in a solution to produce an emulsifier solution. The emulsifier may include one or more of organic and non-organic compounds. The compounds may act as one or more of surfactants, colloids, and drying agents. Further, the emulsifier such as, for example, Gum Arabic may be used according to a ratio of one part of the essential oil to four parts of the emulsifier by weight in some embodiments. In some other embodiments, the emulsifier may be used according to different ratios. Further, the ratio of the essential oil to the emulsifier may affect the solubility of the solid form of the essential oil. Without the proper ratio, when the solid form of the essential oil is mixed with water, the essential oil may fall out of the solution. This problem may be mitigated through the use of heat although the intent, in some embodiments, is to keep the essential oil in solution for the use of addition to cold liquids. Further, in some embodiments, the emulsifier may be agitated and heated in order to aid dissolution and enhance homogeneity of the emulsifier solution.

In some embodiments, the emulsifier used may include surfactants such as naturally occurring soaps, artificial soaps and acids. In some other embodiments, the emulsifier may include colloids such as vegetable solids (e.g. dried and powdered avocado, coconut powder etc.), fibrous foliage, naturally occurring fats such as soy based fats and short chain lipid bilayers. In some embodiments, the emulsifier may include polysaccharides, gum arabic, xanthium gum, gypsum, inert minerals, various salts, various plant based starches, starches, tricalcium phosphate, dextrose flavoring.

Further, in some embodiments, calcium carbonate or other desiccant may be used to absorb moisture and improve flow of granular solids

Subsequently, at step 306, the essential oil solution may be mixed with the emulsifier solution in order to produce a mixed solution. In some embodiments, prior to mixing, the essential oil solution may be heated or alcohol may be added. Further, the mixing may be performed by adding the essential oil solution to the emulsifier solution at a slow rate in order to prevent clumping or lack of full immersion into homogeneous solution. Additionally, the mixing may be performed slowly under continuous heat and agitation. For example, the mixing may be performed using an addition funnel, pipet, pouring, or any other method of controlled slow mixing of the essential oil solution and the emulsifier solution. Further, in some embodiments, the mixed solution may be allowed time to remain in an agitated state to promote homogeneity. However, in some embodiments, this may not be necessary with various solutions which are more prone to cohesive mixing.

Thereafter, at step 308, evaporation of the mixed solution may be performed in order to separate the solvents from the mixed solution. In some embodiments, a rotary evaporator may be used to perform the evaporation. Subsequently, drying of the mixed solution under a vacuum condition may take place to help in the evaporation.

In some embodiments, using the rotary evaporator may collect some of the terpenes and aromatic compounds as condensates. These condensates may also be considered desirable by-products and may be held in an aqueous or alcohol solution for later recovery or consumption.

Further, in some embodiments, the mixed solution may be reduced to a syrup form. Subsequently, the syrup form may be spread out on a non-stick surface and placed into a vacuum oven. Thereafter, the vacuum oven may be maintained at suitable temperature to extract any remaining moisture. As a result, the solid form of the essential oil, such as a hard sheet of the substrate infused with the essential oil and the emulsifying agent may be formed.

In some embodiments, the evaporating may be performed using a falling film evaporator also known as a spray dehydrator, or a spray dryer. One benefit of the falling film evaporator may be that the solid form of the essential oil is the efficiency in drying, and controlled particle size.

At step 310, the solid form of the essential oil may be milled to produce a powder form of the essential oil. For example, the hard sheet may be broken up and placed into a mill in order to granulize the solid. The mill may consist of a variety of apparatus including rotary blade mixing, jet mill, hammer mill, mortar and pestle style mixer, auger type mixer, as well as manual or automatic mixing spreading or crushing of any kind. One objective of the granulation is to render the solid form of the essential oil as particles that range in size from granules to powdered form. In some embodiments, the powdered form may be further subjected to drying if found necessary. For example, the powdered form may be placed back into the vacuum oven or under low pressure evaporating conditions in order to draw out any remaining moisture. Thereafter, the powdered form may be packaged.

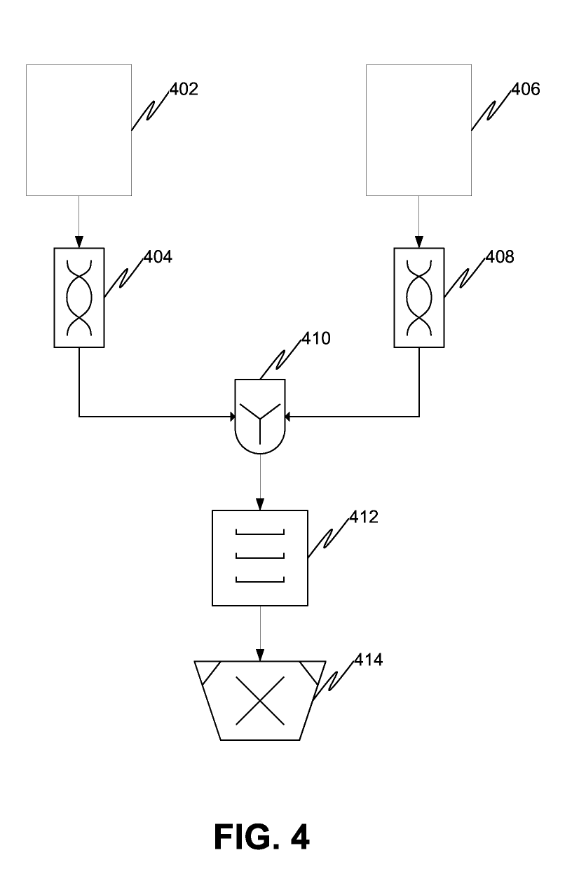

FIG. 4 illustrates an apparatus for preparing a powder form of a cannabinoid. The apparatus may include a container 402 configured to dissolve the Cannabinoid into a carrier solvent of one or more solvents to obtain a Cannabinoid solution. Further, the apparatus may include a mixer 404 configured to mix the Cannabinoid solution to obtain a homogenized Cannabinoid solution. Furthermore, the apparatus may include a container 406 configured to dissolve the one or more emulsifying agents into an aqueous solvent of the one or more solvents to obtain an emulsifier solution. Further, the apparatus may include a mixer 408 configured to mix the emulsifier solution to obtain a homogenized emulsifier solution. Additionally, the apparatus may include a mixer 410 configured to mix each of the homogenized Cannabinoid solution and the homogenized emulsifier solution to obtain a homogenized combined solution. Further, the apparatus may include an evaporator 412 configured to evaporate each of the carrier solvent and the aqueous solvent from the homogenized combined solution to obtain a solid form of the cannabinoid. Furthermore, the apparatus may include a miller configured to mill the solid form of the Cannabinoid to obtain the powder form of cannabinoid.

Although the invention has been explained in relation to the foregoing embodiments, it is to be understood that many other possible modifications and variations can be made without departing from the spirit and scope of the invention.

1. A method of preparing a solid form of a cannabinoid, wherein the solid form of the Cannabinoid is substantially soluble in an aqueous solution, the method comprising:
a. dissolving each of a Cannabinoid and at least one emulsifying agent into at least one solvent to obtain at least one combined solution; and
b. separating the at least one solvent from the at least one combined solution to obtain the solid form of the cannabinoid.

2. The method of claim 1, wherein the separating comprises at least one of evaporating and drying.

3. The method of claim 1 further comprising mixing the at least one combined solution.

4. The method of claim 1, wherein the solid form of the Cannabinoid comprises a powder form of the cannabinoid.

5. The method of claim 3 further comprising converting at least a part of the solid form of the Cannabinoid into a powder form of the cannabinoid.

6. The method of claim 5, wherein the converting comprises milling, wherein an average particle size of the powder form of the Cannabinoid is in a range of 500 microns to 50,000 microns.

7. The method of claim 1, wherein the at least one emulsifying agent comprises at least one of natural gum, β-Glucan and cyclic-β-Glucan.

8. The method of claim 7, wherein the at least one emulsifying agent comprises gum arabic.

9. The method of claim 1, wherein the Cannabinoid comprises at least one of Cannabidiol (CBD), Cannabinol (CBN) and Δ9-tetrahydrocannabinol (THC).

10. The method of claim 1, wherein a weight ratio of the Cannabinoid and the at least one emulsifying agent is in a range of 1 to 16.

11. The method of claim 1, wherein the dissolving comprises:
a. dissolving the Cannabinoid into a carrier solvent of the at least one solvent to obtain a Cannabinoid solution; and
b. dissolving the at least one emulsifying agent into an aqueous solvent of the at least one solvent to obtain an emulsifier solution.

12. The method of claim 11 further comprising mixing each of the Cannabinoid solution and the emulsifier solution to obtain the at least one combined solution.

13. The method of claim 11, wherein a temperature of the aqueous solvent is in a range of 25 to 100 degrees Celsius.

14. The method of claim 11, wherein a temperature of the carrier solvent is in a range of 0 to 100 degrees Celsius.

15. The method of claim 12 further comprising:
a. evaporating the carrier solvent from the at least one combined solution; and
b. evaporating the aqueous solvent from the at least one combined solution.

16. The method of claim 11, wherein the carrier solvent is characterized by each of miscibility with water and ability to dissolve the cannabinoid.

17. The method of claim 16, wherein the carrier solvent comprises alcohol.

18. The method of claim 1, wherein a weight ratio of the Cannabinoid and the at least one emulsifying agent in the solid form of Cannabinoid is in a range of 0.05 to 20.

19. The method of claim 1, wherein the Cannabinoid is comprised in a cannabis extract obtained from a cannabis plant, the method further comprising:
a. winterizing the cannabis extract; and
b. decarboxylating the cannabis extract, wherein the cannabis extract retains a natural terpene profile.

20. A method of preparing a powder form of a cannabinoid, the method comprising:
a. dissolving the Cannabinoid into a carrier solvent of the at least one solvent to obtain a Cannabinoid solution;
b. mixing the Cannabinoid solution to obtain a homogenized Cannabinoid solution;
c. dissolving the at least one emulsifying agent into an aqueous solvent of the at least one solvent to obtain an emulsifier solution;
d. mixing the emulsifier solution to obtain a homogenized emulsifier solution;
e. mixing each of the homogenized Cannabinoid solution and the homogenized emulsifier solution to obtain a homogenized combined solution;
f. evaporating each of the carrier solvent and the aqueous solvent from the homogenized combined solution to obtain a solid form of the cannabinoid; and
g. milling the solid form of the Cannabinoid to obtain the powder form of cannabinoid.
